(19) United States
(12) Patent Application Publication
(10) Pub. No.: US 2008/0275395 A1
(43) Pub. Date: Nov. 6, 2008

(54) MRI-Compatible Temperature-Sensing Catheter

(75) Inventors: David Robbins Asbury, Lake Elsinore, CA (US); Donald Eugene Bobo, Fountain Valley, CA (US)

Correspondence Address: INSKEEP INTELLECTUAL PROPERTY GROUP, INC, 2281 W. 190TH STREET, SUITE 200, TORRANCE, CA, 90504, US

(73) Assignee: Innerspace Medical, inc.

(21) Appl. No.: 11/961,897

(22) Filed: Dec. 20, 2007

Related U.S. Application Data
(60) Provisional application No. 60/876,522, filed on Dec. 22, 2006.

(51) Int. Cl. A61M 25/00

(52) U.S. Cl. 604/113

(57) ABSTRACT

A preferred embodiment includes an MRI compatible catheter that maintains a desired temperature during an MRI procedure. Specifically, the catheter includes a lumen containing sensor wire that is surrounded by an insulating material. This material limits the transfer of heat to the outside of the catheter as the sensor wire heats during an MRI procedure.

### RELATED APPLICATIONS

This application claims priority to U.S. Provisional Application Ser. No. 60/876,522 filed Dec. 22, 2006 entitled An MRI Compatible Temperature-Sensing Catheter which is hereby incorporated by reference.

### BACKGROUND OF THE INVENTION

The measurement of temperature remains an important part of many different medical treatments. Often, catheters are used for temperature measurement, such as a ventricular catheters placed in the brain. Thermistor or thermocouple based temperature sensors are typically used in temperature sensing catheters.

While these measurement catheters are acceptable for many medical purposes, their use within an MRI machine can prove difficult. For example, the wires connected to a temperature sensor increase in temperature when placed in an MRI machine. As the heat from these wires increase, the inner and outer surfaces of the catheter increase in temperature, causing potentially serious injury to the patient.

MRI procedures are typically 15 minutes and typically use a 1.5-3T magnet. During this time, the MR/rf field continually heats the wire based on the strength of the magnet and the length of the wire. FDA guidelines state that the temperature of the exterior of the catheter not rise more than 2° C. during a 15-minute scan.

Designs used to reduce heating of the wires by those skilled in the art include placing coils and capacitors at either end of the wire to neutralize induced currents and insulating the wire with an electrical insulator of sufficient quality to avoid current pathways between wires.

One specific catheter design example can be seen in U.S. Pat. No. 6,829,509, the contents of which are incorporates by reference. This MRI compatible catheter reduces RF heating with incorporated circuitry and materials that address eddy currents and the transfer of electromagnetic energy from one wire to another.

The inclusion of electronics in a catheter is appropriate for particularly expensive devices, especially when an electrode at the distal end of the catheter might be in contact with the body, e.g. pacemaker leads. However, the cost and size of the electronic components used in designs that reduce heating effect are not appropriate for use in some catheters, such as a ventricular catheter used in the brain.

### SUMMARY OF THE INVENTION

Four main catheter embodiments are described according to the present invention that can be used with MRI machines of different magnet strengths. One preferred embodiment reduces heat transfer from the wire to the catheter by stringing the wire through an insulating tube that is then placed in a dedicated lumen. A second preferred embodiment provides a different insulator than the first design. A third preferred embodiment removes heat by a fluid circuit that passes either a fluid or gas (e.g., water or air) past the sensor wire. A fourth preferred embodiment has a removable temperature sensor probe. The sensor is removed prior to an MRI and then replaced.

One objective of each design is to prevent the sensor wires in the catheter from raising the temperature of the catheter body to an unacceptable temperature.

As will be discussed, one preferred embodiment of the present invention includes a dedicated catheter lumen through which the sensor wires pass. The lumen is larger than the lumen used to receive uninsulated wires in the present catheters. Some currently available catheters have a diameter of about 0.02″. In a catheter designed to receive a temperature probe, a 0.03″ lumen is preferred.

Concerning the removable probe embodiment, the probe can be alternatively placed in a manifold attached to a skull bolt. The manifold includes a passage for a catheter (e.g., ventricular catheter) and provides channels through which parameter probes (such as temperature, oxygen, pressure or flow probes) can be passed into the brain. A channel provided expressly for a temperature probe includes a lumen with a closed distal end to allow an unsterile probe to be reinserted after an MRI.

The two embodiments that rely upon insulation require no attention from the hospital staff. The water or air-cooled design requires the staff to provide an air pump or water source such as an IV bag or liquid pump. The removable probe design requires the staff to remove and replace the probe from the catheter. The most appropriate option of the four designs depends upon the strength of the magnet used in the MRI machine.

The first three embodiments of the present invention, insulation, air-cooling or liquid cooling, do nothing to reduce the heating effect of the MRI. They instead reduce heat transfer from the wire to the catheter.

The first two embodiments house the wire in an insulator with properties sufficient to limit the heat transfer from the wire to the catheter so the rise in the temperature of the body preferably does not exceed 4° C., and more preferably 2° C. A third embodiment removes heat by injecting a fluid, either gas or water, past the wire. The fourth embodiment places the sensor and its wires in a removable probe. The probe is removed from the catheter prior to an MRI procedure and reinserted in the catheter after the procedure. The fluid-cooling design and removable probe designs are best suited to be used in a MR machine with a strong magnet and a catheter with long sensor wires.

### BRIEF DESCRIPTION OF THE DRAWINGS

FIG. 1 illustrates an insulating sleeve on sensor wires according to a preferred embodiment of the present invention;

FIG. 2 illustrates a string of insulating beads on sensor wires according to a preferred embodiment of the present invention;

FIGS. 3a and 3b illustrate a fluid-cooled catheter design according to a preferred embodiment of the present invention;

FIG. 4 illustrates a removable temperature probe according to a preferred embodiment of the present invention;

FIG. 5 illustrates a catheter with a guide tube designed to receive a temperature probe according to a preferred embodiment of the present invention;

FIG. 6A illustrates a manifold system within a patient, having multiple lumens for receiving a probe and a catheter according to a preferred embodiment of the present invention;

FIG. 6B illustrates the manifold system of FIG. 6A out of the patient; and

FIGS. 7A, 7B and 7C illustrate various views of the manifold system of FIGS. 6A and 6B

### DETAILED DESCRIPTION OF THE INVENTION

As previously described in this specification, the RF field generated by an MRI machine will heat the wires of a typical catheter. Preferably, the temperature rise of the exterior of an MRI compatible catheter during a typical 15 minute scan time will preferably not exceed 4° C., and more preferably 2° C. The heat generated by the wire is defined by the strength of the magnet and the length of the wire. Longer wires get hotter than shorter wires.

The preferred embodiments of the present invention allow a temperature-sensing catheter to be exposed to the field of an MR machine. Since catheter length and therefore the length of the sensor wires can vary considerably from one catheter to another, different preferred embodiments may be appropriate with different catheter designs.

Generally, the first two preferred embodiments provide an insulator between the wire and the catheter. The third preferred embodiment removes the heat generated by passing a cooling fluid by the wire during the scan. The fourth preferred embodiment removes the wire from the catheter during an MRI scan.

Note that the catheters in which a temperature sensor is placed may have more than one lumen for other medical purposes and procedures. For simplicity, the drawings omit extra lumens.

Also, note that the term wire will be used to describe the two wires of a temperature sensor. While the probe of the present invention is specifically referred to as a temperature probe, it should be understood that the present invention is particularly applicable to any sensor type that includes conducting wires (e.g., an oxygen probe). Thus, the probe as described in the specification may be any probe/sensor that uses electrically conductive wires.

Insulating Sleeve

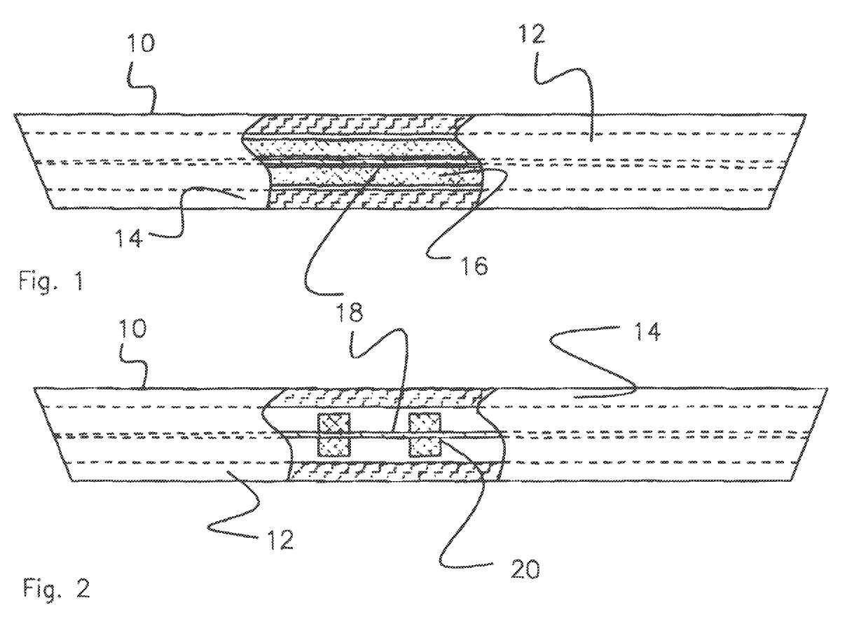

Referring to FIG. 1, a catheter 10 with a wire lumen 12 is formed by a wall 14 that encircles the lumen. A sensor wire 18 is positioned within an insulating tube 16 that insulates the wire 18 from the lumen wall 14.

Preferably, the insulating tube 16 is flexible enough not to overly stiffen the catheter 10, can withstand the temperature of the wire and includes a low coefficient of heat transfer. One example insulating material is polyimide, which is ductile, has a particularly low coefficient of heat transfer and is available for use in thin-wall tubes.

The thermocouple and thermistor wires are preferably 0.005″ in diameter and form a twisted wire set including electrical insulation of about 0.014″ in diameter. The diameter of the insulating tube 16, affects the overall catheter diameter.

Preferably, the tube 16 has an ID of 0.020″ and an OD of 0.026″ which balances the size of the lumen 12 and the efficacy of the insulation. If the catheter lumen wall is 0.004″, the tube 16 of these dimensions provides a wall thickness of 0.007″. If used in a ventricular catheter, the example insulating tube 16 will reduce the heat transferred from the wire 18 to the catheter 10 by over two thirds.

The basis of the heat reduction of the previous example is as follows. Present silicone catheters typically have a wall thickness about 0.02″. The conductivity of polyimide is ⅙ that of silicone. The 0.007″ wall of tube 16 therefore has about the same insulating quality as a 0.042″ silicone tube wall. When the insulating value of the silicone wall is added to that of the polyimide wall of the tube 16, the insulating effect is equal that of a silicone wall 0.062″ thick or about 3 times that of a silicone tube alone. Over time, the temperature of the wire 18 will increase due to the reduction in heat transfer from wire 18 to the wall of the catheter 10. The higher temperature of the wire 18 will reduce the net effect of the insulating tube 16, so improvement may be less than a ⅓ reduction. The lumen diameter required to receive the 0.026″ OD insulating tube 16 is about 0.03″. Such a lumen is about twice as large as a lumen required to string a wire set without a thermal insulation sleeve or tube 16.

Insulating Beads on a Wire

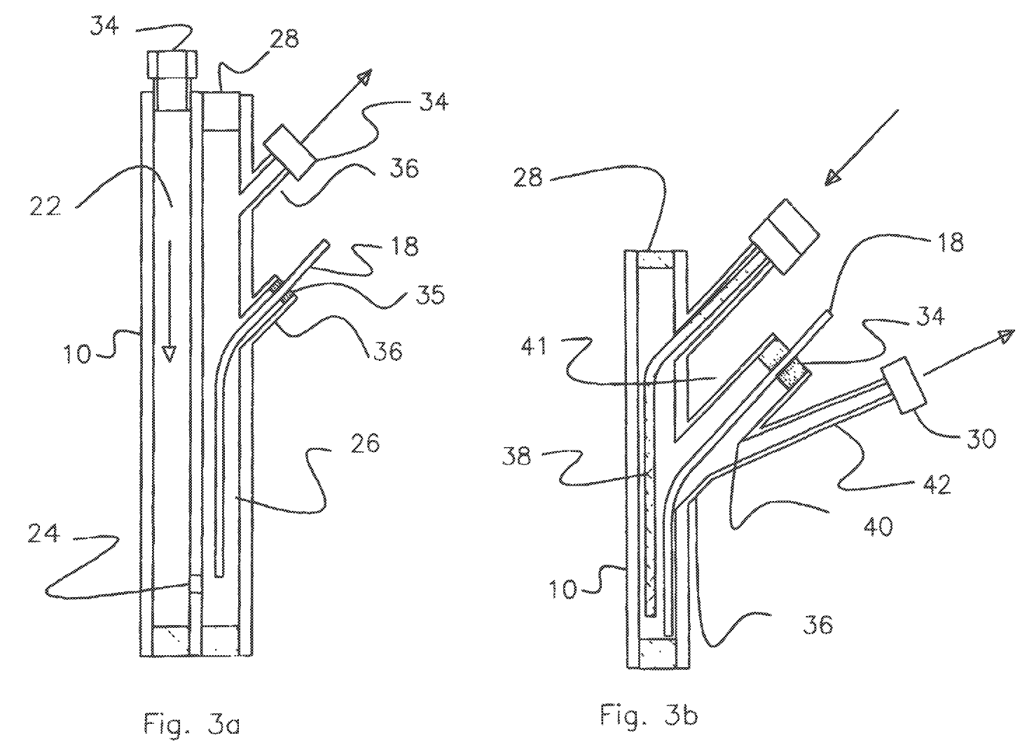

FIG. 2 illustrates another preferred embodiment of an insulating arrangement according to the present invention in which short insulating tubes, segments or beads 20 are placed along the wire 18. Preferably, the beads 20 are longitudinally spaced along the wire at intervals that keep the catheter wall 12 from contacting the bare wire 18 if the catheter 10 is bent to the extent that it forms a 0.25″ radius.

In one specific example, beads 20 have a length of 0.05″, are composed of polyimide and are fixed on the wire 18 at intervals of 0.150″. The air gap between the beads has a lower K factor than polyimide and thus reduces overall heat transfer. In cal/(cm)(sec)(° C.), the conductivity of air is 6.9×10⁻⁷ vs. 3.7×10⁻⁴ for polyimide, a three orders of magnitude difference. This construction can be used in an MRI with a stronger magnet and/or a catheter with a longer wire than the preferred embodiment of FIG. 1.

Fluid Cooled Catheter

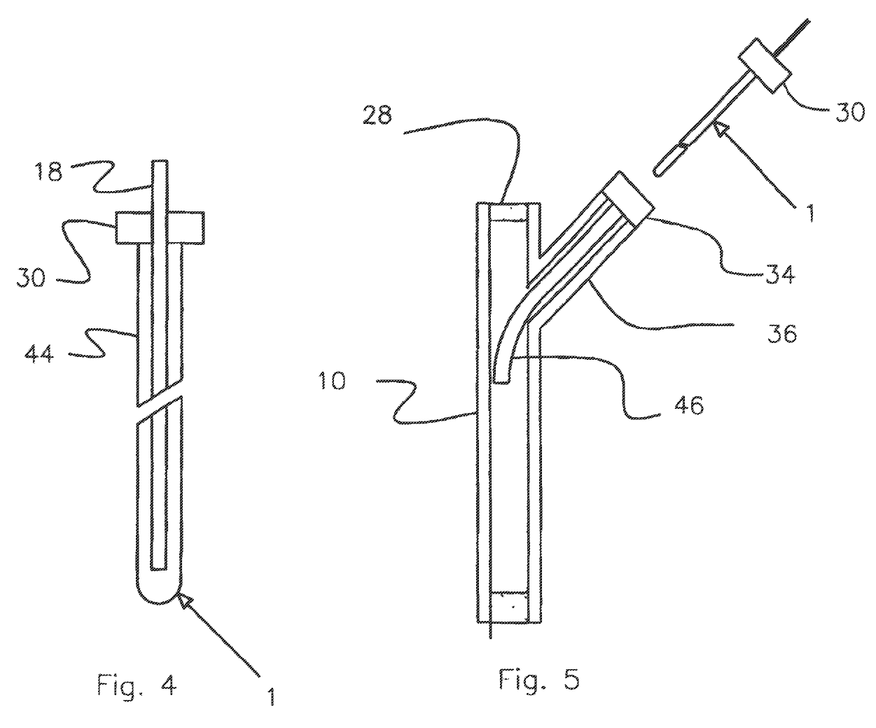

In another preferred embodiment, a cooling fluid or gas (e.g., water or air) is passed over or adjacent the wire. FIG. 3a illustrates a two-lumen construction in which an inflow lumen 22 delivers (e.g. via a pump) a cooling fluid through a crossover 24 to an outflow lumen 26 and ultimately to an exit in a transition tube 36. The wire 18 is located in the outflow lumen 26 and is thereby cooled by the cooling fluid. The wire 18 is fixed to a transition tube 36 by an adhesive 35. Both the lumens are closed where needed by a plug 28. The inlet lumen has a luer connector 34 on its proximal end that mates with a similar connector on a fluid source line. A transition tube 36 carries the wire out of the catheter to a connector, not shown. The cooling fluid exits out of a transition tube. The transition tube has a luer connector 34 that can mate with a take-away line, not shown. Thus, the cooling fluid can be delivered over the wire 18 during an MRI procedure and thereby maintain the temperature of the catheter.

FIG. 3b illustrates a single fluid lumen embodiment that uses a fluid delivery tube 38 that extends the length of the wire lumen and delivers fluid to the bottom of the lumen. The fluid cools the wire 18 as it flows out the lumen. A transition tube 36 has a Y 40 that separates the wire 18 from the outflow fluid. The cooling fluid exits the lumen through an outflow line 42 that has a luer fitting 30. The wire 18 exits the upper leg 41 of the Y where the wire 18 is bonded to an interior of the lumen of the catheter by an adhesive 34.

Removable Temperature Probe

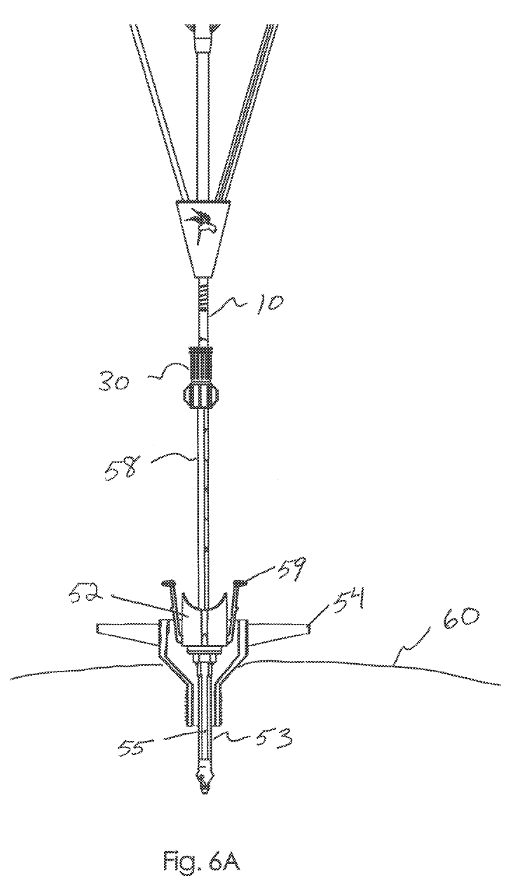

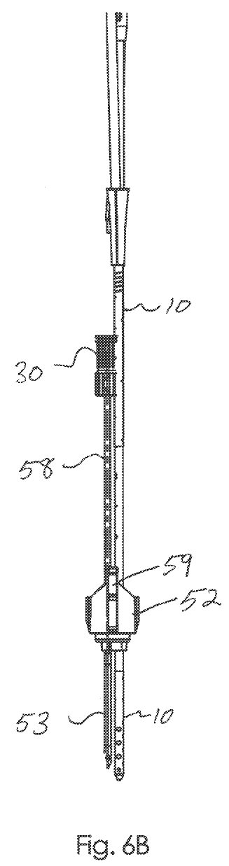

FIG. 4 illustrates a preferred embodiment of a removable temperature probe 1. Preferably, the temperature probe 1 can be removed prior to an MRI procedure, thereby eliminating any temperature increase of the wires 18. Alternately, any of the previously described insulation or cooling embodiments (such as those seen in FIGS. 1-3b) can be used with the wire 18 of probe 1, allowing the user to choose an appropriate probe 1 for a specific MRI machine. The removable temperature probe 1 allows the device to be used in any strength MRI machine if the other components of the catheter 10 (e.g., the catheter of FIG. 5) are non-magnetic.

The wire 18 of the probe 1 is located in a probe housing 44. The distal end of the housing is a closed hemisphere. The wire 18 passes through a probe connector 30 (e.g., a luer fitting) and is held in place by an adhesive.

FIG. 5 illustrates a catheter 10 with a lumen and transition tube 36 designed to receive the temperature probe 1. Both of the lumen ends are closed with a plug 28. The transition tube 36 has a transition tube connector 34 that mates with the probe connector 30 on the temperature probe 1. A guide tube 46 is preferably attached to the proximal end of the transition tube 36. The curvature of the guide tube 46 and its low friction properties enable the temperature probe 1 to be inserted into the lumen of the catheter 10. Inserting the probe into a 0.025″ diameter lumen without the attributes of the guide tube may be problematic as the bend radius required to enter a 0.025″ lumen is quite small. When the probe is fully inserted in the catheter, the guide tube connector 34 and the probe connector 30 are joined.

Preferably, the probe housing 44 is composed of polyimide, as it is available in very thin wall tubes and has a low coefficient of friction. In one example, the polyimide probe housing 44 is 0.01″ ID×0.015″ OD. The lumen diameter of the catheter is 0.03″. A guide tube 46 is integrated into the provided port. It serves two functions. The entry of the probe 1 into the catheter 10 requires that the probe make a very sharp turn into a very small lumen of the catheter 10. The curved section of the guide tube 46 causes the probe 1 to curve as it makes the transition from the guide tube to the catheter lumen. The guide tube 46 also provides a surface with a low coefficient of friction. Catheter materials are somewhat grabby and can make the insertion of the probe into a small diameter lumen difficult.

In operation, the catheter 10 is implanted within a patient and the removable probe 1 is placed within the catheter 10, coupling the transition tube connector 34 and the probe connector 30. Prior to an MRI procedure, the transition tube connector 34 and the probe connector 30 are uncoupled and the probe 1 is removed. Once the MRI procedure has been performed, the probe 1 can be reintroduced into the catheter 10 and the transition tube connector 34 and the probe connector 30 can be coupled once more.

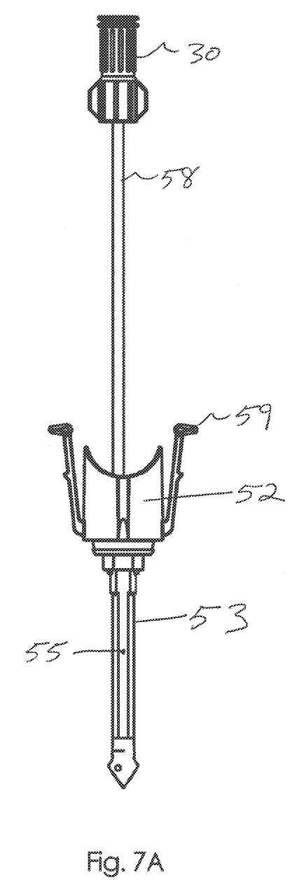

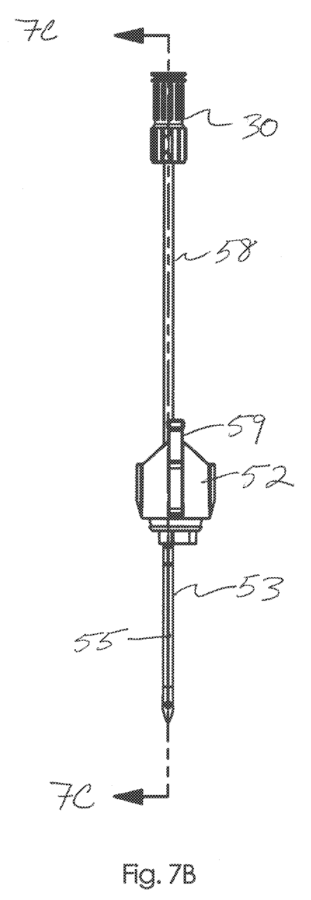

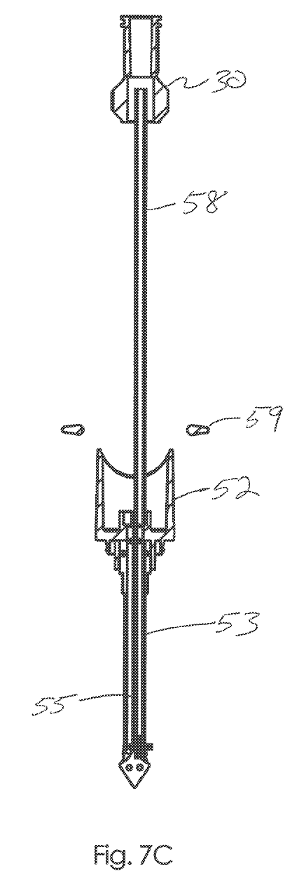

FIGS. 6A through 7C illustrate a preferred embodiment similar to the previously described preferred embodiment, further including a manifold 52 that snaps into or couples to a bolt 54 via biased locking tabs 59. The bolt 54 includes a portion implantable within a patient (e.g., a patient's skull) and a passage therethrough. The manifold 52 includes multiple passages on its proximal end that feed into a multi-lumen extension 53 connected on its distal end. In other words, the manifold 52 “funnels” or directs different probes, tubes or other devices into the multi-lumen extension 53 via individual passages. Preferably, the manifold 52 also includes a catheter passage that allows a catheter to pass through the manifold 52, and ultimately into the patient, but not into the multi-lumen extension 53. In this respect, a probe 1 can be advanced into a patient without being sterilized since the multi-lumen extension 53 provides a barrier between the patient and the probe 1.

The multi-lumen extension 53 is an elongated tubular member that may be a separate extension that couples directly to a distal side of the manifold 52 or optionally is unitary with the manifold 52. Alternately the multi-lumen extension 53 may be a distal part of a catheter 10 (i.e., similar to the catheter shown in FIG. 5).

Preferably, a Touhy-Borst fitting holds a catheter 10 (e.g., a ventricular catheter shown in FIGS. 6A and 6B) to the manifold 52 and therefore to bolt 54, allowing the catheter 10 to pass into the patient. The manifold 52 preferably includes several pigtails (e.g., flexible tubes, one of which is shown as pigtail 58) that each connect to a passage on the proximal end of the manifold 52. The temperature probe 1 can be inserted into the brain through one pigtail and other sensors, such as an oxygen sensor can be inserted into other pigtails. For the sake of clarity, only pigtail 58 is shown in FIG. 6A-7C. This pigtail 58 includes a probe connector 30 (e.g., a luer connection) on its proximal end. Alternately, the probe 1 may be passed directly into the manifold 52 without the use of a pigtail 58.

The temperature probe 1 is inserted into a pigtail 58 and is secured by the probe connector 30. The distal end of the probe 1 passes through a passage of the manifold 52 and into a lumen 55 of the multi-lumen extension 53. When fully inserted, the temperature sensor of the probe 1 resides in the patient (e.g., a patient's brain) but is separated from actual contact with the patient by the closed multi-lumen extension 53. The temperature probe can be inserted and removed as needed. Preferably, the lumen 55 is closed at a distal end (i.e., the end inserted into the patient), however the lumen 55 may also be open within the multi-lumen extension 53.

Since the pigtail 58 is connected to the manifold 52, any pushing, pulling or other force exerted on the pigtail 58 or probe 1 will be transferred to the manifold 52 and the bolt 54, instead of to the catheter 10, as may occur in the preferred embodiment of FIGS. 4 and 5.

In operation, the bolt 54 is implanted within the patient (e.g., into patient's skull 60 in FIG. 6A). Next, the catheter 10 is inserted through the Touhy-Borst of the manifold 52, through the bolt 54 and into the patient (e.g., the brain). The manifold 52 is then connected to the bolt 54. The probe 1 is inserted into the pigtail 58, through the manifold 52 and into the lumen 55 of the multi-lumen extension 53. The probe connector 30 is coupled to the probe 1 to maintain the position of the probe 1. Prior to an MRI procedure, the probe connector 30 is uncoupled from the probe 1 and the probe 1 is removed from the catheter 10. Once the MRI procedure is complete, the probe 1 can be reintroduced into the pigtail 58 as previously described.

Alternately, the pigtail 58, the manifold 52 and the multi-lumen extension 53 are composed of an insulating material as previously described in this specification (e.g., polyimide). In this respect, the probe 1 can be left within the patient during an MRI procedure.

The first two embodiments of FIGS. 1 and 2 are convenient in that they require no involvement from the MRI staff. The third and fourth embodiments may require that the staff prepare the catheter prior to the MRI but may be less expensive than the first two embodiments.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. An MRI compatible catheter comprising:
a catheter body having a lumen;
a sensor wire disposed within said lumen; and
an insulating tube disposed around said sensor wire.

2. The MRI compatible catheter of claim 1, wherein said sensor wire makes up a thermocouple.

3. The MRI compatible catheter of claim 1, wherein said sensor wire makes up a thermistor.

4. The MRI compatible catheter of claim 1, wherein said insulating tube comprises polyimide.

5. An MRI compatible catheter comprising:
a catheter body having a lumen;
a sensor wire disposed within said lumen; and
a plurality of insulating members disposed on said sensor wire.

6. The MRI compatible catheter of claim 5, wherein said plurality of insulating members are comprised of polyimide.

7. The MRI compatible catheter of claim 5, wherein said plurality of insulating members are beads.

8. The MRI compatible catheter of claim 5, wherein said plurality of insulating members are tubes.

9. An MRI compatible catheter comprising:
a catheter body having a first lumen in communication with a second lumen; said first lumen coupled to a supply of cooling fluid and said second lumen having an exit for said cooling fluid;
a sensor wire located in said second lumen so as to be contacted by cooling fluid during inflow of said cooling fluid to said catheter body.

10. The MRI compatible catheter of claim 9, further comprising a crossover passage between said first lumen and said second lumen.

11. The MRI compatible catheter of claim 10, wherein said crossover passage is located at a distal end of said catheter.

12. An MRI compatible catheter comprising:
a catheter having a lumen;
a first passage in communication with said lumen and a cooling fluid supply;
a sensor wire at least partially disposed in said lumen and positioned through a second passage that leads to an outside of said catheter;
a third passage providing an exit for said cooling fluid from said lumen.

13. The MRI compatible catheter of claim 12, further comprising a fluid tube having a first end coupled to said first passage and a second end positioned near a distal end of said lumen.

14. A removable catheter comprising:
a catheter body having a lumen and a first passage in communication with said lumen;
a probe removably disposed in said first passage; and
a user-actuated connection between said first passage and said probe.

15. The removable catheter of claim 14, further comprising a tube having a first end coupled to an opening of said first passage and a second end positioned within said lumen; said second end being closed.

16. A method of using a catheter comprising:
providing a catheter comprising a sensor wire;
placing a catheter within a patient;
supplying a cooling fluid to said catheter;
operating an MRI machine on said patient;
limiting a temperature rise of said catheter to about 4° C.

17. The method of claim 16, further comprising limiting said temperature rise of said catheter to about 2° C.

18. A method of using a using a catheter comprising;
placing said catheter within a patient;
removing a probe from a lumen said catheter;
performing an MRI procedure on said patient;
replacing said probe within said lumen of said catheter.

19. The method of claim 18, wherein said probe senses temperature.

20. A method of using a probe comprising:
placing a bolt within a patient;
providing a manifold member connected to an elongated extension member;
coupling said manifold member to said bolt;
placing a probe through a passage of said manifold and into a lumen of said elongated extension member;
removing said probe from said passage;
performing an MRI procedure on said patient;
replacing said within said passage of said manifold member.

21. The method of claim 20, wherein said placing a temperature sensing probe through a passage of said manifold and into a lumen of said elongated extension member further comprises introducing said probe into a pigtail member.

22. The method of claim 20, wherein said elongated extension member comprises multiple lumens.

23. A system for removably placing a probe within a patient comprising:
a bolt member implantable within a patient;
a manifold connectable to said bolt member; said manifold comprising a first passage;
an elongated extension in communication with said first passage; and
a probe connectable to at least one of said plurality of passages.

24. The system of claim 23, further comprising a flexible lumen having a first end connected to said first passage and a second end selectively connectable with said probe.

25. The system of claim 23, wherein said manifold and said elongated extension are composed of polyimide.

26. The system of claim 23, wherein said manifold comprises a plurality of passages.

27. The system of claim 23, wherein said manifold includes a second passage configured to direct a catheter directly into a patient.
